(19) United States
(12) Patent Application Publication
(10) Pub. No.: US 2023/0173003 A1
(43) Pub. Date: Jun. 8, 2023

(54) COMPOSITION FOR THE TREATMENT OF RADIO-INDUCED ORAL MUCOSITIS

(71) Applicant: MAGI EUREGIO SCS, Bolzano (BZ) (IT)

(72) Inventors: Matteo Bertelli, San Felice del Benaco (BS) (IT); Elena Manara, Dro (TN) (IT); Paolo Enrico Maltese, Rovereto (TN) (IT); Stefano Cecchin, Vicenza (IT); Giuseppe Marceddu, Bolzano (IT); Kyrylo Anpilogov, Valfabbrica (PG) (IT); Stefano Paolacci, Tivoli (RM) (IT); Daniele Malacarne, Pergine Valsugana (TN) (IT); Teresa Zelante, Corciano (PG) (IT); Stefano Giovagnoli, Perugia (IT); Debora Caricato, Lecce (IT); Leonardo Brizzi, San Giustino (PG) (IT)

(73) Assignee: MAGI EUREGIO SCS, Bolzano (BZ) (IT)

(21) Appl. No.: 17/941,310

(22) Filed: Sep. 9, 2022

(30) Foreign Application Priority Data
Sep. 9, 2021 (IT) 102021000023381

(51) Int. Cl. A61K 35/747; A61K 9/00; A61K 47/10; A61K 47/38; A61P 1/02

(52) U.S. Cl. CPC A61K 35/747; A61K 9/006; A61K 47/10; A61K 47/38; A61P 1/02

(57) ABSTRACT

A composition, in particular in gel form, includes Lactobacillaceae reuteri to be used in the prevention and/or treatment of oral mucositis, particularly radio-induced oral mucositis. In its gel form, the composition also includes: a polyoxy-alkylene copolymer, in particular a polyoxy-ethylene-propylene copolymer, preferably with the general formula HO[CH₂CH₂O]ₓ[CH₂CH(CH₃)O]_{y}[CH₂CH₂O]_{z}H, where preferably x=99, y=67, and z=99; a mucoadhesive polymer, preferably cellulosic in nature, in particular carboxymethyl cellulose; and one or more stabilizers, preferably chosen from sugars such as sucrose, trehalose, mannitol, sorbitol and glucose, sodium ascorbate, very preferably the sugar is sucrose. Treatments of other mucositis with the composition, such as intestinal leakage, are conceivable. A procedure to produce the composition in gel and lyophilized form is described.

### REFERENCE TO AN ELECTRONIC SEQUENCE LISTING

The contents of the electronic sequence listing (6847-0110PUS1.xml; Size: 21,170 bytes; and Date of Creation: Sep. 2, 2022) is herein incorporated by reference in its entirety.

### CROSS REFERENCE TO RELATED APPLICATIONS

This non-provisional application claims the benefit under 35 U.S.C. § 119(a) to Patent Application No. 102021000023381, filed in Italy on Sep. 9, 2021, which is hereby expressly incorporated by reference into the present application.

### FIELD OF THE ART

The invention concerns a composition for preventing and treating radiation-induced oral mucositis and a process for producing said composition. Methods for the prophylaxis and treatment of radiation-induced oral mucositis are described.

### STATE OF THE ART

Radiation-induced oral mucositis (RIOM) is considered one of the major side effects of radiation therapy for head and neck cancers. The term mucositis refers to damage to healthy non-tumor tissues, lasting between 7 and 98 days, which affects with acute inflammation the oral, tongue, and pharyngeal mucosa with the recall of immune cells and the release of cytokines, chemotactic factors, and growth factors [1]. RIOM can progress to severe physical obstruction to food and fluid ingestion, resulting in weight loss and septic complications [2]. RIOM is a very common clinical entity, present in approximately 80% of patients with head and neck cancers undergoing radiotherapy [3]. Risk factors for radio-mucositis include: concomitant chemotherapy (CT), poor oral hygiene, substandard nutritional status, lack of antibiotic use in the early stages of the disease, and cigarette smoking [4]. RIOMs are entities that underlie many different symptoms and clinical pictures, such as pain in the oral cavity, dysphagia [1, 5]. The quality of life of patients suffering from RIOMs is reduced so dramatically that, in 11-16% of patients, it leads to discontinuation of the cancer disease treatment and, thus, failure to resolve the malignancy [6].

Recent studies have confirmed that the pathogenesis of radio-induced mucositis consists of four distinct phases: an initial inflammatory and/or vascular phase, an epithelial phase, an ulcerative/infectious phase, and a healing phase [7]. During the initial inflammatory phase, after exposure to radiation treatment, there is direct damage to cellular DNA, resulting in the release of reactive oxygen species (ROS) by epithelial and endothelial cells, fibroblasts, and macrophages [8]. Pathogenesis and pathogenetic mechanism have been described by several authors [8, 9, 10, 11]. The establishment of micro-coagulative phenomena and post-actin neutropenia, which further facilitate the colonization and subsequent infection by Gram-negative bacteria and yeasts, is described. Microbial exotoxins, particularly bacterial, further worsen the inflammatory reaction by increasing the release of IL-1β, TNF-α, and nitric oxide [10].

RIOMs usually develop within about two weeks from the beginning of radiotherapy. The diagnosis of radiotherapy-induced mucositis is basically clinical. There are many different scales for estimating the clinical impact of RIOMs [12]. Early recognition of the development of oral mucositis is essential to undertake therapy [13].

The occurrence of mucositis is greatly limited by the use of new radiation techniques, such as intensity modulated radiation therapy (IMRT) [14].

One of the key preventive elements to avoid the occurrence of lesions is proper oral hygiene. However, excessive oral hygiene could be responsible for the depletion of resident microbial flora and consequently generate dysbiosis, expanding the chances of overgrowth of opportunistic germs and infectious complications.

The use of keratinocyte growth factor (KGF) is definitely an effective weapon for preventing the development of RIOM and it has already been approved by FDA as a preventive treatment [15].

Other preventive interventions include: the use of amifostine, an antioxidant and cytoprotective agent administered subcutaneously and intravenously [16, 17]; the use of intra-oral radiation shielding devices [18, 19]; the use of low-energy helium-neon lasers before radiation treatment [20].

Treatment of mucositis, which should be started as early as possible, has two main purposes: to reduce the symptomatic picture that greatly impacts the patients' quality of life and to shorten the convalescence time, facilitating healing and limiting complications. Mucositis therapy exploits both locally applied agents and pharmacological prescriptions administered systemically.

Topical therapy relies on local analgesics, antioxidants, and anti-inflammatory agents that allow both reduction of the localized inflammatory state and algic symptoms, which cause considerable discomfort and significantly impact the patients' quality of life. Among the most commonly used topical agents are: glycerritinic acid-, povidone iodine-, or sodium hyaluronate-based gels; L-glutamine; manganese superoxide dismutase; local anesthetics such as diphenhydramine, xylocaine, lidocaine, diclonine hydrochloride; Vitamin A and Vitamin E. When the lesion is advanced and significantly compromises the patient's nutrition and quality of life, or if local therapy is no longer sufficient to control the symptomatology, it is necessary to resort to pharmacological devices that act on the body as a whole. Systemic-acting agents, orally, intramuscularly, or intravenously administered, include: cyclooxygenase 2 (COX-2) inhibitors; N-acetylcysteine; minor analgesics and opioids; azelastine; systemic corticosteroids; and antibacterial and antifungal agents (in cases of bacterial or fungal infections). [21-31]

Reduced salivary flow, loss of the mucosal barrier, and dysbiosis resulting from radiation exposure can induce overgrowth of commensal polymicrobial populations. Under these conditions, germs normally present as resident microorganisms can give infection. [32]

Should the initial lesion become complicated with infection, whether bacterial or fungal, it will be necessary to implement analgesic and anti-inflammatory therapy by administering antimicrobial agents. Antibiotic agents, such as brilacidin, have shown significant benefit in both preventing the onset of mucositis and reducing the degree of its severity and septic complications [33].

The infections that complicate the clinical picture, especially in immunocompromised patients, are very often caused by fungi. The pathogen family most frequently isolated in oral cavity infections after radiation treatment is Candida spp. In 80% of cases, C. albicans is involved; in the remaining cases, the infections are caused by C. glabrata and C. tropicalis.[14, 34] Therefore, it turns out to be of paramount importance, in the presence of this complication, to use systemically antifungal agents such as fluconazole and clotrimazole. However, the same agents have also shown remarkable efficacy in a preventive sense: early administration ensures not only the avoidance of Candida spp. infection in immunocompromised patients, but also a reduction in the incidence and severity of RIOMs. [3, 35]

The oral microbiome is considered one of the most complex microbial systems in our body, second only to the intestinal microbiome. Thanks to the Human Oral Microbiome Database (HOMD), it has been possible to understand the exact composition and possible variations of microbial populations in the various diseases that can affect the oral cavity. [36]

The oral microbiome is composed mainly of five phyla: Firmicutes, Bacterioidetes, Proteobacteria, Actinobacteria, and Spirochaetes. These five major families comprise about 94% of all detected microorganisms. These bacteria, thanks to their production of metabolites that are useful to our body (post-biotic) and to the immunological tolerance that makes them commensal, ensure tissue homeostasis. [36]

The loss of balance among these species, termed dysbiosis, can cause the onset of a rich array of oral pathologies, from dental caries to local and systemic infections. Fluctuations in the predominant species of the oral microbiome have been demonstrated in various pathologies.

The pathogenesis of mucositis is multifactorial and based on a set of different factors that cooperate in order to cause the onset of the lesion. This “puzzle” does not recognize precise etiological factors; rather, it is a patchwork of concomitant factors that determine the motive for the onset of mucositis.

Several research groups have attempted to demonstrate variations in the cluster of microorganisms present before and during radiation treatment, in order to ascertain what kind of effect radiation therapy had on the microbiome.

In a 2012 study, the microbiome of the oral cavity of eight patients with neoplasms of the head-neck district undergoing radiation treatment was analyzed. These analyses revealed not only that, during radiation therapy, there was a rarefaction of the rich bacterial community, and consequently a decrease in operational taxonomic units (OTUs), but also that the relative prevalence of cariogenic bacterial species, compared with healthy controls, such as Streptococcus, Veillonella, and Actinomyces. [37]

By sequencing the 16S subunit gene of bacterial ribosomal RNA, Zhu et al.'s research group in 2017 assessed the biodiversity of the oral microbiome in patients undergoing radiation therapy for nasopharyngeal carcinoma. Analyses showed a close correlation between cumulative radiation dose, mucositis severity and qualitative-quantitative changes in the microbial community. In the most severe mucositis researchers found a relative prevalence of bacterial genera like Phenylobacterium, Acinetobacter, Burkholderia, Sphingomonas, Azospirillum, Rhizobium, Hydrogenophaga, Paracoccus and Nocardioides. These genera would, therefore, appear to be positively associated with the development of medium to severe mucositis, while the genera Leptotrichia and Peptostreptococcus seem to be depleted under such conditions, consequently negatively associated. [38]

More recently, in 2018, Hou et al. attempted to encode the changes the microbiome undergoes during radiation treatment. The oral microflora showed remarkable changes during radiation treatment: the genera Pseudomonas, Treponema, and Granulicella were significantly increased during radiation therapy, while Prevotella, Fusobacterium, Leptotrichia, Campylobacter, and Prevotalla were markedly decreased in irradiated patients compared to healthy controls. [39]

In light of this, the reduction in the biodiversity of the oral microbiome seems positively correlated with the amount of radiation absorbed by the oral cavity and with the possibility of developing RIOMs, as well as their severity. Qualitative-quantitative depletion of the oral microflora is greatest during the ulcerative phase. Such a major alteration implies a loss of microbiota-host tissue homeostasis, inevitably affecting the ecosystem symbiosis as well. This dysbiotic mechanism could explain the process whereby, precisely during the ulcerative phase, there is the highest probability of developing infections, especially fungal infections. Controlling the altered distribution of microbial populations seems to be an excellent expedient to prevent the spread of yeasts and the establishment of complicated candidiasis pictures. An increase in xerostomia, fungal infections, and the presence of Lactobacilli spp. are closely correlated with a reduction in oral microbiome biodiversity and in ecosystem symbiosis. [40] Fungal infections are one of the most frequent and also most disabling complications that follow the onset of mucositis. This occurs because normal mucosal defenses, such as salivary IgA production, are lost, and because the subsequent dysbiosis results in the loss of normal symbiotic conditions between commensal microorganisms and host tissue.

During the ulcerative phase, when the physiological symbiosis is lost, an increased presence of Lactobacillus reuteri has been demonstrated. This particular microorganism is in effect a protector of oral homeostasis, capable of safeguarding the mucosa from the spread of yeasts such as Candida spp.

Indeed, it has been shown by researchers of the University of Perugia that this microorganism, particularly present in the gastric and small intestinal mucosa, is capable of degrading tryptophan into indole derivatives with potent post-biotic, pro-regenerative, immunogenic and tolerogenic activities. In particular, the indoles produced can bind the aryl hydrocarbon receptor (AhR). Activation of the AhR, especially at the level of small intestinal lymphocytes, stimulates the IL-22-dependent response, which is capable of both enhancing mucosal defense by inducing the proliferation of basal epithelial cells, and of combating fungal spread. The same activated receptor is able to elicit the anti-inflammatory response through the expansion of regulatory T lymphocytes (Treg) and the production of anti-inflammatory interleukins, such as interleukin 19 (IL-10). [41]

The role of Lactobacillus reuteri in defending against the complications of mucositis is still not entirely clear, but it is certainly a prime example of how the microbiome is often able to protect our body from anything that attempts to undermine its homeostasis.

Guo and his research group analyzed the murine intestinal microbiome and the possible relationship between fluctuations in it with acute radiation injury.

Acute radiation syndrome (ARS) is defined as that pathological condition resulting from partial or total body exposure to ionizing radiation. The onset of this syndrome is related to a poor hematopoietic bone marrow response and to mucosal epithelial cell death in the gastrointestinal tract.

Scientists noticed that a small subset of SPF C57BL/6 mice, about 5-15%, when exposed to high total body radiant doses (about 9.2 Gy), not only recovered very rapidly from ARS but were also marked by longer survival. These mice were termed “elite survivors,” and their microbiome was analyzed and compared with that of mice that did not survive the radiant dose. Fecal transplants and a peculiar method called “dirty cage-sharing” between “elite survivors” mice and naive mice were used in order to “transfer” the gut microbiome from the former to the latter. Microbiome analysis, by sequencing the gene for the 16S subunit of ribosomal RNA on fecal samples, showed a significant prevalence, as of day 7 after the start of irradiation, of Lachnospiraceae, Enterococcaceae and Lactobacillaceae in elite survivors, compared with controls.

In “elite survivors” mice and mice that shared the cage (and consequently the microbiome) with them, more intense bone marrow proliferation (by histologic staining with Ki67) and better splenic extramedullary hematopoiesis were found. The more effective hematopoiesis accounts for the shorter recovery and longer survival of these mice after radiation exposure.

The assumption that the above three bacterial species have radioprotective action can be attributed to their ability to produce active metabolites: post-biotics. The most important active metabolites include short chain fatty acids (SCFAs). Both Lachnospiraceae and Lactobacillaceae are capable of producing significant amounts of SCFAs, such as proprionate and butyrate. These metabolites are associated with reduced levels of produced proteins as a result of genomic damage, such as γH2AX, p53, and 53BP1. The reduced production of these proteins, together with decreased levels of intra-medullary ROS, is indicative of reduced genomic and oxidative stress and consequently increased cellular resistance to radiation damage. [42]

The role of the microorganisms in our microbiome is still not entirely clear, however, their importance in maintaining the homeostasis of various tissues and organs is crystal clear.

### DISCLOSURE OF THE INVENTION

The object of the invention is to propose an alternative composition to what is known in the state of the art to prevent and/or treat radiation-induced oral mucositis. Further aim is to develop a related procedure for the prevention/prophylaxis and treatment of radiation-induced oral mucositis, to propose formulations that allow effective application of the composition, and a related delivery device.

In a first aspect of the invention, the object is achieved by a composition, particularly in the form of a gel, comprising Lactobacillaceae reuteri to be used in the prevention and/or treatment of oral mucositis, particularly radio-induced oral mucositis, preferably by oral application. An advantageous oral application is by spray.

The inventors identified L. reuteri as a particularly suitable lactobacillus in the treatment of mucositis and transferred the function of lactobacillus in the intestine to the oral cavity, a step that, also in light of the state of the art, was not obvious, given the very different conditions of the two environments. A way was found to exploit the role played by Lactobacillaceae in the gut also in the oral cavity, particularly because it was possible to identify a specific formulation that allows the lactobacillus to be applied effectively in the oral cavity. The genus lactobacillus is highly represented in the intestinal environment, but poorly in the oral environment; changing the environment of the bacterium therefore was not an obvious approach. In this regard, it is particularly advantageous to have the composition in the form of a gel, preferably a thermogel, that is, a thermoreversible gel. The term “thermoreversible” means a gel that can be reused by heating it once it has been thickened. This is important to make the gel storable at low temperatures, such as 4° C., to protect lactobacilli, and then usable at the temperatures present in the oral cavity. The gel is a two-phase elastic colloidal material, consisting of a dispersed liquid that is incorporated into the solid phase. The liquid “dwells” in the structure consisting of the solid, which in turn takes advantage of the surface tension of the liquid so as not to collapse. In this regard, a preferred embodiment of the invention provides that the composition further comprises:
(b) a polyoxy-alkylene copolymer, particularly a polyoxy-ethylene-propylene copolymer, preferably having the general formula HO[CH₂CH₂O]ₓ[CH₂CH(CH₃)O]_{y}[CH₂CH₂O]_{z}H, where preferably x=99, y=67 and z=99;
(c) a mucoadhesive polymer, preferably cellulosic in nature, especially carboxymethyl cellulose; and
(d) one or more stabilizers, preferably selected from sugars such as sucrose, trehalose, mannitol, sorbitol and glucose, sodium ascorbate, very preferably the sugar is sucrose.

The thermoreversible gel is not only sprayable, but also capable of carrying a probiotic buccally. The composition, particularly in its thermogel form, is suitable for oral use.

A preferred polyol is commercially available under the name Pluronic® F-127. The use of different sugars, even combinations of sugars, is conceivable; the best results have been obtained with sucrose. Sugars are important for bacterial growth and protection of microorganisms during a possible lyophilization process, they also act as cryostabilizers. Carboxymethyl cellulose is known as a food additive, identified with code E466. These components do not have an adverse effect on the viability of the contained bacteria.

To further enhance the efficiency of the composition to treat mucositis in a preferred embodiment of the invention the stabilizer comprises tryptophan. Advantageously, it is present in the form of polymeric microparticles suitable to release the tryptophan over a prolonged period of time. As the system hydrates, the tryptophan is released and metabolized by the bacteria. Such formulations are preferably stored as powders, e.g. lyophilized, so as not to trigger release too early. It is conceivable to add to such a microparticle formulation a proportion of free tryptophan which is immediately utilized when the gel is reconstituted and administered. In animals (mammals, in particular mice) tryptophan concentrations of 100 nM to 1 μm are generally conceivable.

In a particularly advantageous embodiment of the invention, the composition comprises the copolymer, particularly of the general formula above, in an amount between 20 and 25% (w/v), preferably 21% (w/v), the mucoadhesive polymer, particularly carboxymethyl cellulose, in an amount between 0.1 and 0.5% (w/v), preferably 0.3% (w/v), and sugar, particularly sucrose, in an amount between 5 and 15% (w/v), preferably 8% (w/v).

The abovementioned components in their respective concentrations gave the best results. Preferably, L. reuteri is present in the composition at a concentration of about 1×10⁸ to 1×10⁹ CFU/ml, specifically at a concentration of about 1×10⁹ CFU/ml.

The liquid component of the composition is advantageously made by a phosphate buffered saline. Other liquids are conceivable, particularly buffers, known to the person skilled in the art.

The proposed gel, in addition to making it possible to evaluate the effect of pro- and post-biotics in the clinical course of mucositis, is capable of carrying, without killing, beneficial microorganisms. The gel is able to carry bacteria, particularly Lactobacillus reuteri, while maintaining their viability. Moreover, said gel is easily stored and easy to administer. The proposed gel is lyophilizable, and its reconstitution by supplementing the liquid or aqueous component, allowing it to gel only at temperatures proper to the oral cavity (˜30-34° C.).

A second aspect of the invention relates to a lyophilized product obtained from the composition according to the invention, particularly from the composition in gel form, by lyophilization, hence a lyophilized product comprising L. reuteri and advantageously the other indicated components.

A third aspect of the invention relates to a production process of the composition according to the invention, comprising the following steps:
(I) preparation of a phosphate buffered saline (PBS) and solubilization of a bacterial suspension of L. reuteri in order to obtain a desired concentration, preferably verified by absorbance with calibration according to the straight line

$$y = \frac{x - 0. 006}{0.00005} ,$$

and preferably avoiding mechanical stress;
(II) solubilization of desired amounts of a mucoadhesive polymer, preferably cellulosic in nature, in particular carboxymethyl cellulose, and a polyoxy-alkylene copolymer, in particular a polyoxy-ethylene-propylene copolymer, preferably with the general formula HO [CH₂CH₂O]ₓ[CH₂CH(CH₃)O]_{y}[CH₂CH₂O]_{z}H in the bacterial suspension, wherein it is preferable to first supplement the mucoadhesive polymer and then the polyoxy-alkylene copolymer at temperatures around 0° C., with subsequent storage of the system at about 4° C. for about 12 hours to achieve complete solubilization of the polymer; e
(III) adding one or more stabilizers, in particular sugars, preferably after solubilization of the polymers.

The individuation of the suitable composition and of the suitable process for its production to be able to apply the gel in an efficacious way in the oral cavity has been accompanied by testing temperatures and gelification times, the mucoadhesion strength and the sprayability of the composition, tests described below.

In a very preferred embodiment of the invention, the process further comprises the step of
(III) lyophilization of the system obtained in step (II), in particular at about 4° C. for about 24 hours; and optionally the step of
(IV) reconstitution of the gel by supplementing the lyophilized with an aqueous component, in particular a phosphate buffered saline.

A lyophilized is easily storable and supplementable at low temperatures; the reconstitution of the aqueous composition by supplementing the lyophilized with the aqueous component is possible at low temperatures. The resulting cold solution has viscosities that are suitable to be sprayed with a classical nonpressurized dispenser into the oral cavity, and only in situ at higher temperatures, above 20° C., the composition gels by covering the mucosa of the oral cavity with good adhesive characteristics.

Another aspect of the invention relates to a prophylaxis method of oral mucositis, in particular the radiotherapy-induced one, involving the application of the gel according to the invention, as specified above, one day before the radiotherapy treatment, on the same day of the radiotherapy treatment, and on the second and third day after the radiotherapy treatment, thus on days −1, 0, 2, and 3.

An additional aspect of the invention involves a kit comprising:
(i) as first component, a lyophilized according to the invention or a composition according to the invention in lyophilized form;
(ii) as second component, an aqueous liquid, in particular a phosphate buffered saline; and optionally
(iii) a dispenser, particularly a non-pressurized dispenser.

The kit can be stored in a refrigerator, and, at the time of need, the lyophilized is supplemented with the aqueous component and can be administered to the patient via the dispenser.

One aspect of the invention relates to a method for testing the efficacy of treatment with L. reuteri that involves analysis of gene expression of the components involved in 3-IALD signaling, such as Cyp1A1, AhR, IL-10, and IL-22; of R-spondin 1 expression; of AhR systemic expression; and/or expansion of regulatory T lymphocytes.

In a further aspect of the invention, the proposed composition is intended to treat leaky gut i.e. intestinal leakage.

### DESCRIPTION OF PREFERRED EMBODIMENT EXAMPLES

### Brief Description of the Drawings

FIG. 1 illustrates the set-up of the gel with probiotic: (a) definition of the calibration line for quantitative spectrophotometric evaluation of L. reuteri; (b) colony count on plate culture with MRS Agar (according to De Man, Rogosa and Share) to assess gel viability; (c) determination of best probiotic gel composition.

FIG. 2 illustrates the mouse model for mucositis induction and for treatment with L. reuteri-enriched gel: (a) timing of the protocol; (b), (c), (d), (e) examined mice populations.

FIG. 3 illustrates the application of the mouse model; (a) development of oral mucositis in mice, arrow indicates macroscopically visible mucositis; (b) administration of the gel by probe.

FIG. 4 illustrates the clinical evaluation of the mouse model: a) evaluation of the mice's weight gain following mucositis induction; b) evaluation of the mice's weight loss following mucositis induction.

FIG. 5 illustrates the evaluation at day 8 of the survival of the different groups of mice.

FIG. 6 illustrates the histologies of the mice's tongues excised after sacrifice: a) NAIVE, histology of tongues of naive mice; b) CTIOM GEL, histology of tongues of control mice, treated with gel only; b) CTIOM GEL+, histology of tongues of mice treated with L. reuteri.

FIG. 7 illustrates the immunological response of lingual tissue at day 5, after CTIOM induction: (a) gene expression of IL-10; (b) gene expression of IL-22; (c) gene expression of Cyp1A1; (d) gene expression of AhR.

FIG. 8 illustrates the analysis of R-spondin 1 expression in the tongues of: naive mice, control mice, and mice receiving gels with L. reuteri.

FIG. 9 illustrates the gene expression, evaluated at the spleen level: (a) IL-10; (b) IL-22; (c) AhR; (d) Cyp1A1; (e) R-spondin 1.

FIG. 10 illustrates the growth of organoids, as seen under the microscope: (a), (b) image of the well where the organoids were cultured as seen under a bright-field microscope; (c), (d) image of the organoids after 11 days of culture under phase contrast microscope, maximum diameters of about 730 μm and 460 μm, respectively; (e) image of an organoid after 11 days of culture under phase contrast microscope. All images were taken at the Department of Pathology and Immunology, University of Perugia.

FIG. 11 illustrates the histologies of lingual organoids: (a) H&E (Hematoxylin and Eosin)—hematoxylin eosin (HE) staining; (b) CKHMW—immunolabeling for high molecular weight cytokeratins; (c) p63—immunostaining for p63.

FIG. 12 illustrates the evaluation of the transcriptional response of organoids following infection with L. reuteri, analysis of R-spondin 1 expression.

FIG. 13 illustrates the clinical evaluation of the mouse model for RIOM: evaluation of the mice's weight development following mucositis radio-induction.

### MATERIALS AND METHODS

### Description of the Mouse Model

For the mouse model, wild type C57/BL6 mice, eight to nine weeks old and 20 to 25 grams of initial body weight, were used. The animals were maintained under standard environmental conditions, at an ambient temperature of 23±2° C. and humidity of about 60±10%. The mice were provided free access to food and water. The experimental design was sent to OPBA and the Ministry of Health and approved under Project No. 725/2019 PR.

### Induction of Acute Chemotherapy Induced Oral Musitis (CTIOM)

Mucositis induction was achieved by administering a chemotherapeutic agent, 5-fluorouracil (5-FU), and an ulcerating agent, acetic acid (AA). The use of 5-FU is likely to ensure the occurrence of the immunosuppressive state that is typical of both chemotherapy and radiation treatment. Acetic acid, on the other hand, is necessary to induce local damage at the level of the oral mucosa: being a burning agent, it causes the loss of the superficial epithelium, leading to the appearance of the characteristic ulcers of CTIOM. 5-FU was administered at a dosage of 50 mg/kg of body weight by intraperitoneal injection. The administration of this chemotherapeutic agent was done 5, 3 and 1 days before time 0 and on the first day. On day 0, anesthetic premedication with pentobarbital and 2% xylazine hydrochloride was applied, then a gel containing xylazine was applied for the purpose of achieving localized anesthesia at the site of acid exposure. Both lingual margins, right and left, were treated with a cotton swab that had been previously infused with 25 μl of 50% acetic acid. The one-time administration of acetic acid, varying in duration from 30-60 seconds, is the local noxious stimulus essential to trigger the onset of mucositis. To remove any residual or excess acetic acid, the treated mucosa was thoroughly cleansed through the use of cotton swabs infused with sterile saline solution. For the whole duration of the experiment, the mice were given free access to properly shredded food mixed with water.

Then, on the fifth day, the mice were sacrificed and their organs and other elements useful for laboratory analysis (such as tongue, cheek and spleen) were removed through aseptic procedures. The samples thus obtained were immediately placed in a solution with 10% formalin, to ensure their adequate fixation and preservation until analysis.

The parameters that were evaluated in the experiment are:
The weight of the mice, measured before each administration, an extremely reliable parameter for determining the presence of mucositis and dysphagia. In fact, weight loss is a very sensitive indicator of the mice's inability to enterally feed themselves.
Food consumption while caged.
Il consumo del cibo in gabbia.
Early death before scheduled sacrifice.

After inducing mucositis, the mice were administered the (thermo)gel according to the invention, a gel that solidifies at certain temperatures and includes probiotic agents.

### Preparation of the (Thermo)Gel

The gel in question was obtained by mixing precise amounts of excipients capable of giving the compound the abovementioned characteristics. To determine the right ingredients and their exact concentrations, several formulations were developed, with constituents that differed in both quality and quantity. Initially, 3 different formulations containing sucrose, treaolose, or glucose, essential sugars for bacterial growth and for the protection of microorganisms during the lyophilization process, were tested. In addition to the saccharide component there were carboxymethylcellulose (CMC), polyvinylpyrrolidone (PVP), ascorbic acid, and pluronic F-127; however, PVP has been shown to inhibit bacterial growth, while polyol promotes it. Ascorbic acid is a potential stabilizer. Other tested compositions and components significantly reduce bacterial viability. A concentration between 1×10⁸ and 1×10⁹ CFU/ml of Lactobacillus reuteri was also added to these ingredients. It is preferable to use a concentration of 1×10⁹ CFU/ml.

To set up the gel, a polymer dispersion was prepared in a dispersing medium, consisting of phosphate buffered saline (PBS). First, in a suitable volume of PBS (14.52 ml), the bacterial suspension was solubilized in an amount of 480×10⁻³ ml, in order to obtain a final concentration of 320×10⁶ CFU/ml. To limit any reduction in the viability of the bacterial strain, the procedure was performed in its entirety under a fume hood, and the mechanical stresses to which the bacteria might have been subjected were reduced. Subsequently, the polymers were appropriately weighed and solubilized directly into the bacterial suspension, according to precise w/v % concentrations.

Polymer solubilization required a special procedure: initially, PVP and CMC were integrated, then pluronic F-127 was cold dissolved in the solution. Briefly, the polymer was added to the solution of PVP and CMC placed in ice bath gradually, to disfavor aggregation processes. After that, the system was stored at 4° C. for about 12 hours, achieving complete solubilization of the polymer. The suspension was then stored at 4° C. until its use, to prevent gelification.

The viability of the bacteria included in the gel was then assessed by seeding the gel on MRS Agar plates incubated at 37° C. with 5% CO₂.

### Determination of Gelification Temperature

Gelification temperature (Tgel) to optimize gel composition was determined by rheological investigation, using a Viscotech rotational rheometer (Reologica AB instruments) in a flat-to-flat configuration equipped with a Peltier system for temperature control. The analysis was performed in oscillatory mode, selecting a gap of 0.5 mm. In addition, shear stress values of 1 Pa and shear rate of 1 s⁻¹ were set, while the temperature range used is 15-37° C. (heating ramp rate of 1° C./min). Samples (0.7 ml) were carefully applied to the lower plate of the rheometer and allowed to equilibrate for a time of 2 min before each analysis.

### Determination of Gelification Time

The sol-gel transition time (tgel) to optimize gel composition was determined by visual inspection, using the inversion method. An amount equal to 0.5 ml of the sample was placed in a vial. This was placed in contact with a water bath, thermostated to 32° C. The gelification time was defined as the time when no movement of the sample is observed after tipping the tube.

### Determination of Mucoadhesive Force

An indirect method was performed to determine the adhesion characteristics of the system under investigation against a simulated gastro-intestinal mucus. The analysis was performed, using a rheometer, in oscillatory sweep stress mode, with a flat-to-flat configuration and selecting a gap of 0.6 mm. The test was performed at a frequency value of 1 Hz and a temperature of 32° C., while the selected stress range is 0.009 to 1.5 Pa. Viscoelastic behavior was evaluated to determine the binding ability between the gel and the simulated mucosal fluid. In order to evaluate the mucoadhesive capacity and to obtain a prediction of a hypothetical administration into the buccal cavity, an assay was set up that could allow simulated in vivo administration, adhesion of the system to the mucosa, strength, and permanence time of the gel. An amount equal to 0.5 ml of the sample was placed on the lower plate, while 0.3 ml of the simulated fluid was layered on the upper plate. The test was started after 5 min, contact time between the two components in order to ensure sufficient binding. The stress value at which a change in viscoelastic behavior was observed was considered as the force required for detachment of the system from the mucus.

### Spray Ability Test

Considering that this formulation is intended to be administered through a non-pressurized spray device, a method was developed to evaluate the efficiency and homogeneity of formulation dispensing, which may be hindered by the viscosity and by the gelification process. The formulation was placed in a device, consisting of amber glass bottles (5-20 mL) and equipped with a nozzle that allows dispensing amounts of 70/100/140 μl. The device was placed at a distance of 6 cm from a round paper sheet, with an area of 23 cm² that was divided into eight equal sectors, useful for evaluating the homogeneity of spray distribution.

The threshold time for complete gel administration was determined by keeping the filled device, previously stored in a refrigerator, at room temperature for different time intervals before device actuation. Incomplete device emptying and nozzle blockage were used as indicators.

In addition, in order to evaluate the effect of stress caused by dispensing on the bacteria, viability after dispensing was then assessed.

### Lyophilization

The lyophilization process was used to transform the thermoreversible bacterial suspension into a dry powder, in order to increase the storage stability of the product. The process was conducted by initially slow freezing the liquid suspension, starting from the storage temperature of 4° C. Lyophilization was conducted for 24 hours. At the end of the process, the powdered product was stored away from moisture at 4° C. In order to ensure stability during the freezing process, cryostabilizers, such as trehalose, mannitol, and sucrose, were tested from the point of view of the effect on rheological behavior and bacterial viability retention after the lyophilization process. Additional additives, such as antioxidants, are considered to increase stability during storage.

### Stability Analysis

Product stability was determined on liquid and lyophilized samples, both stored at a temperature of 4° C. Lyophilized samples were also kept away from moisture. Viability and rheological tests were carried out at set times over six months, according to international standards.

### Induction and Evaluation of Acute RIOM

Mice were have been used to reproduce the onset of RIOMs through a standardized protocol. [43]

Initially, mice were anesthetized with ketamine, in order to not compromise the irradiation process because of uncalculated movements.

In addition, for the very same purpose, the mice were placed supine in ad hoc containers that allow to immobilize the mice while at the same time emitting radiation directed only at the animals' head and neck. A custom-made physical shield has been created: this is a lead lid, 6 mm thick, which can be applied above the containers so as to shield the body of the mice and leave only the head-neck district uncovered.

Ionizing radiation was delivered to the mice through a laboratory animal-specific X-ray irradiator. The irradiation was delivered with a constant dose rate of 1.325 Gy/min. The total administered dose was 6 Gy in a single fraction.

After irradiation, the mice were placed in a heated holder and then placed back in their cages. The mice were given free access to water and soft food for the duration of the experiment. The parameters assessed during the experiment were body weight and survival.

### Sample Histology

The tissues (cheek, tongue, and spleen) were removed and immediately fixed with 10% neutral buffered formalin (Bio-optica) for 24 hours. The tissues were dehydrated, soaked in paraffin, and then sectioned into 3-4 μm thick slices. Finally, the slides were stained with hematoxylin and eosin (HE) and analyzed.

RNA Extraction and Quantitative Polymerase Chain Reaction (qPCR)

RNA was extracted from the harvested organs at the end of the mucositis induction procedure. RNA extraction was performed by using TRIzol (Invitrogen, Milan, Italy), a trade name for guanidinium thiocyanate, useful for lysing cells, degrading cellular proteins, and blocking DNA-ase and RNA-ase activity.

For the subsequent retro-transcription, cDNA synthesis-kit from BioRad (Milan, Italy) was used. Real-time PCRs (CFX96 Touch™ Real-Time PCR Detection System) were set using SYBR Green master mix (Agilent Technologies, Milan, Italy). In this study, the polymerase chain reaction consisted of 45 cycles of amplifications divided into three distinct steps:
Denaturation phase, at 95° C. for 1 minute,
Annealing phase, lasting 1 minute with a specific temperature for each primer (Table 1),
Extension phase, at 72° C. for 30 seconds.

All reactions were repeated at least three times, to ensure reproducibility of results. β-actin was chosen as the housekeeping gene for the normalization of quantitative RNA determination. The ΔΔct formula was used to calculate the relative quantity of target genes using BioRad software:

$$R = \frac{{( {E}_{target} )}^{Δ Cp target ( contol - sample )}}{{( {E}_{reference} )}^{Δ Cp reference ( contol - sample )}}$$

Primers' sequences for transcript amplification by PCR from mouse organs are given in Table 1 below.

TABLE 1

| | | | Annealing | |
|---|---|---|---|---|
| | | | temperature | Length |
| Primer | Sequences | | (° C.) | (bp) |
| β-Actin | Sense → | SEQ. 1 | 59.4 | — |
| | Antisense → | SEQ. 2 | 58 | |
| AHR | Sense → | SEQ. 3 | 60 | 344 |
| | Antisense → | SEQ. 4 | 60.6 | |
| CYP1A1 | Sense → | SEQ. 5 | 60 | — |
| | Antisense → | SEQ. 6 | | |
| IL-22 | Sense → | SEQ. 7 | 57.7 | 101 |
| | Antisense → | SEQ. 8 | | |
| IL-22R | Sense → | SEQ. 9 | 57.8 | — |
| | Antisense → | SEQ. 10 | | |
| MUC-2 | Sense → | SEQ. 11 | 58 | 129 |
| | Antisense → | SEQ. 12 | | |
| R-Spondin | Sense → | SEQ. 13 | 57 | 110 |
| | Antisense → | SEQ. 14 | | |
| TNF-α | Sense → | SEQ. 15 | 57 | — |
| | Antisense → | SEQ. 16 | 58 | |

### In Vitro Experimentation: The Use of Organoids

### Organoid Preparation

Organoid preparation is based on the extraction of AdSCs (adipose-derived stem cells=mesenchymal stem cells from adipose tissue) from organs, particularly the tongue, excised from adult mice after sacrifice.

The extracted tongues were then fractionated into small parts, so that the enzymes suitable for digesting the cartilaginous part of the tongue could penetrate, and were thoroughly washed with cold PBS.

The resulting material was then immersed in 50 IU/ml dyspase at 37° C. with 5% CO₂ for 60 minutes, to remove the parenchymatous component of the organ and release the stem cells. After further washing with PBS, the remaining matter was placed in 10 ml of chelating buffer and placed in a shaker at a temperature of 4° C. for 15 minutes. The chelating buffer used consisted of: sodium citrate (27 mM), sodium hydrogen phosphate (5 mM), sodium chloride (94 mM), potassium dihydrogen phosphate (8 mM), potassium chloride (1.5 mM), D-sorbitol (55 mM), and sucrose (44 mM). Next, the resulting mixture, containing the stem cells, was passed through a 40 μM filter. Doing so resulted in a portion of already filtered material, or “fraction 1”, and a portion of unfiltered material, or “fraction 2”, which will be placed in an additional 10 mL of chelating buffer. Both fractions were then centrifuged at 400 rpm at a temperature of 4° C., and the supernatant was replaced with 1 mL of DMEM F12. DMEM F12 consists of F12 with the addition of: Glutamax (2 mM), Hepes (10 mM), penicillin (100 U/ml), streptomycin (100 μg/ml) and N-acetylcysteine (1 μM). An amount of Matrigel (gelatinous protein mixture secreted by Engelbreth-Holm-Swarm (EHS) mouse sarcoma cells), proportional to the material obtained, was added to the remaining material for each fraction. A drop of 50 μl of Matrigel per well was then deposited in a plate previously preheated at 37° C., given the peculiarity of this scaffold to solidify at room temperature. The plate with Matrigel was, then, placed at 37° C. in an incubator with 5% CO₂ for 20 minutes, to ensure the gel solidification. Once solidified, 250 μl of DMEM F12 (Dulbecco's Modified Eagle Medium) medium, supplemented with the necessary substances to ensure the growth and proliferation of adult stem cells, was added to the scaffold. The supplements added to the basic medium are: R-spondin (1000 ng/ml), noggin (100 ng/ml), B27 supplement (1×), N2 supplement (1×), mEGF (50 ng/ml) and Y27632 (10 μm).

The organoids were grown in an incubator at 37° C. with 5% CO₂, and culture medium was changed every 2 to 4 days.

Preparation of L. reuteri and Infection of Organoids

Lactobacillus reuteri (L. reuteri) is a bacterium belonging to the Lactobacillaceae family, normally found as a commensal microorganism in the gastro-intestinal tract of humans and animals. Many clinical studies have shown how adequate administration of L. reuteri can benefit human health, which is why it is currently considered as a probiotic organism. It grows in a specific culture medium for the isolation of lactobacilli, called De Man, Rose and Sharp (MRS) medium. L. reuteri is commercially available and readily accessible through various suppliers. An example strain that can be used is Lactobacillus reuteri ATCC BAA-2837™, available in the ATCC (American Type Culture Collection) bank; in addition, suitable strains, which have the same metabolism and activation of endogenous receptors, can be isolated by the person skilled in the art from the gastro-intestinal tract, i.e., from clinical isolates of L. reuteri.

### Preparation of MRS Broth for Culture in Liquid Medium

MRS broth for the growth of L. reuteri was prepared in laboratory, and contained the following ingredients: peptone, meat extract, yeast extract, D(+)-Glucose, dipotassium hydrogen phosphate, sodium acetate trihydrate, triammonium citrate, magnesium sulfate heptahydrate, manganous sulfate tetrahydrate.

The final pH was found to be 6.2±0.2 at 25° C. Then 51 g of it was dissolved in 1 liter of distilled water, to which 1 ml of Tween 80 (Sigma-Aldrich, Cat. No. P8074), a detergent that facilitates its growth, was added. The whole was then subjected to boiling in order to completely dissolve the medium. Finally, containers were filled with it and sterilized in autoclave at 121° C. for 15 minutes.

### Preparation of MRS Agar Plates for Colony Culture

MRS Agar plates had the following composition: universal peptone, meat extract, yeast extract, D(+)-Glucose, dipotassium hydrogen phosphate, diammonium hydrogen citrate, sodium acetate, magnesium sulfate, manganous sulfate, Agar.

The result was a final pH of 6.5±0.2 at 25° C. Then 61.15 g of the compound were dissolved in distilled water and 1 ml of Tween 80 (Sigmar-Aldrich, Cat. No. P8074) was added, thus increasing the volume to 1000 ml. The whole was boiled to completely dissolve the medium and then autoclaved at 121° C. for 15 min.

Determination of the Calibration Line for the ATCC Strain of L. reuteri

In order to make an accurate quantitative evaluation of L. reuteri cultures, a calibration line was created for measurements by spectrophotometry.

A suspension was prepared from the mother (ATCC: American Type Culture Collection) of isolated colonies of L. reuteri in 10 ml of MRS culture broth, which were left to incubate overnight. The following day, the following dilutions were prepared in final volume of 1 ml in sterile eppendorf:

Table 2 shows the dilutions to be used for determining the calibration line of the ATCC strain of L. reuteri.

TABLE 2

| Dilution | Volume |
|---|---|
| White (MRS broth only) | 1 ml |
| 1:2 | 1 ml |
| 1:10 | 1 ml |
| 1:10² | 1 ml |
| 1:10³ | 1 ml |
| 1:10⁴ | 1 ml |
| 1:10⁵ | 1 ml |
| 1:10⁶ | 1 ml |
| 1:10⁷ | 1 ml |
| 1:10⁸ | 1 ml |

The OD600 (optical density measured at 600 nm) of each of the dilutions was measured. The dilutions were then seeded onto MRS Agar plates. Counting was performed the following day.

Infection of Organoids with ATCC Strain of L. reuteri

After at least 5 days of culture and growth of the organoids, they were exposed to L. reuteri. Infection was carried out using 1×10³ of exponentially growing L. reuteri per well, containing 1-2 murine tongue organoids. Prior to infection, it is necessary to remove the complete medium containing 1% penicillin and streptomycin, that could limit bacterial growth. After overnight exposure at 37° C.+5% CO₂, RNA extraction was done. The above amount was applied in vitro at a 1:10 ratio with the cells present in the organoid. In contrast, 1×10⁸ to 1×10⁹ CFU/ml is used for the in vivo mucositis model.

RNA Extraction from Organoids

To extract RNA from the organoids, it is necessary to collect cell aggregates from the wells and to process them properly in order not to lose the scarce genetic material present.

Initially, the plate containing the organoids was centrifuged at 1200 rpm for 5 minutes. The supernatant culture medium, if present, was removed from the wells; however, it was retained for possible cytofluorometric analysis for evaluation of the cytokine profile. The organoids were then treated with 500 μl of cell recovery medium and incubated at 4° C. for 10 to 15 min. The contents of each well were then transferred into sterile 2-mL eppendorfs and the procedure already described for RNA extraction was applied.

The primers used for amplification of the transcripts by PCR on murine organoids are listed, together with their respective sense and antisense sequences and annealing temperatures, in Table 3 below:

TABLE 3

| | | | | Annealing | |
|---|---|---|---|---|---|
| | | | | temperature | Length |
| Primer | Sequences | | | (° C.) | (bp) |
| β-Actin | Sense | → | SEQ. 1 | 59.4 | — |
| | Antisense | → | SEQ. 2 | 58 | |
| CYP1A1 | Sense | → | SEQ. 5 | 60 | — |
| | Antisense | → | SEQ. 6 | | |
| SEQ. 7IL-22 | Sense | → | SEQ. 7 | 57.7 | 101 |
| | Antisense | → | SEQ. 8 | | |
| R-Spondin | Sense | → | SEQ. 13 | 57 | 110 |
| | Antisense | → | SEQ. 14 | | |
| TNF-α | Sense | → | SEQ. 15 | 57 | — |
| | Antisense | → | SEQ. 16 | 58 | |

### Histology and Immunohistochemistry of Organoids

Just like in vivo-harvested tissues, organoids can be processed to obtain histological and immunohistochemical images in order to obtain a qualitative assessment of cellular composition.

The organoids were, therefore, separated from Matrigel by using cell recovery solution, and their paraffin sections were stained with hematoxylin/eosin (HE) dye. Hematoxylin stains negatively charged cell components, which are mainly localized at the level of the nucleus, such as nucleic acids, membrane proteins, cell membranes, and elastin, in blue/violet. In contrast, eosin colors positively charged cellular components, such as many cellular proteins, mitochondrial proteins, collagen fibers, cytoplasm, and extracellular substances, in red/pink. The observation was made at magnifications of 400× and 200×. Immunostaining with anti-keratin K5 and K14 antibodies was then performed, which showed positivity in cells of the outermost layer of the organoids. Immuno-staining for p63, a squamous cell differentiation antigen that specifically marks basal, suprabasal, and parabasal cells within the tongue epithelium, was positive within the nucleus of the organoid cells.

### Results of the Mouse Model

### Set-Up of the Probiotic Gel

For the evaluation of the efficacy of the gel containing L. reuteri in murine mucositis, a probiotic gel was created (as described in the “Materials and Methods” section), which was administered during the mucositis induction process.

The various attempts in determining the most suitable components of the gel and their relative concentrations led to the understanding that, of all the ingredients used, two in particular affect the growth of the bacterium. In fact, polyvinylpyrrolidone has been shown to inhibit the growth of Lactobacillus reuteri, while pluronil F-127 promotes it.

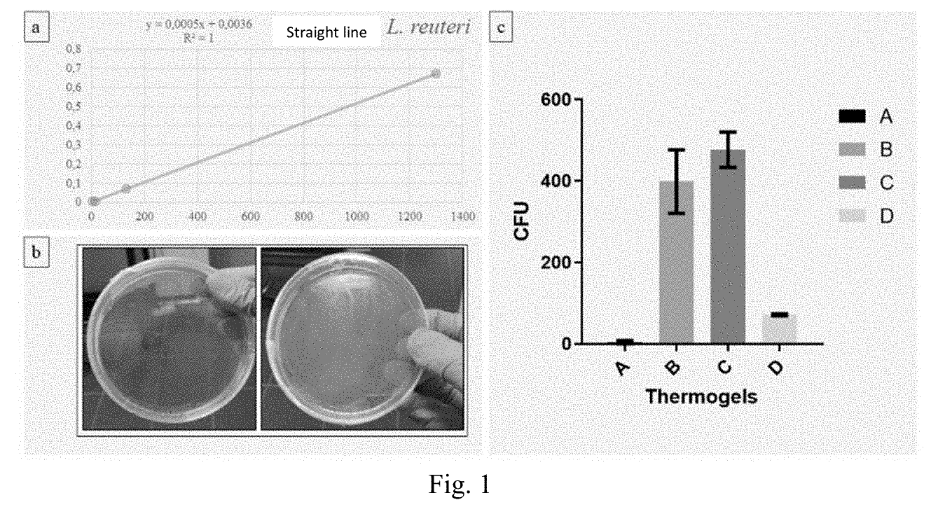

Many formulations have been tested (FIG. 1c), however, the composition that has been found to be optimal at conveying Lactobacillus reuteri, simultaneously ensuring the viability of the microorganism and the necessary characteristics of the gel, is composed of Pluronic F-127, carboxymethylcellulose (CMC) and sucrose, mixed according to precise percentage amounts (FIG. 1c, thermogel C; Table 4).

TABLE 4

| | Percentage |
|---|---|
| Polymer | amount (p/v %) |
| Pluronic F-127 | 21% |
| Carboxymethylcellulose (CMC) | 0.3% |
| Sucrose | 8% |

Moreover, the gel contains precise amounts of L. reuteri, as determined by spectrophotometric techniques and colony counts in classical cultures on MRS Agar. For proper use of the spectrophotometric technique, it was necessary to draw up a calibration line, which made it possible to limit errors in bacterial concentration counts on liquid culture medium (MRS broth). Through the calibration line (FIG. 1a), it was possible to obtain the following equation, which was useful in normalizing bacterial counts by absorbance:

$$y = \frac{x - 0.006}{0.00005}$$

To ensure proper storage and easy administration, the gel was subsequently lyophilized. At the time of use, it is necessary to reconstitute the dried material by adding an aqueous medium. A phosphate buffered saline restoration solution is then added to the lyophilized. The volume of PBS to be supplemented depends on the initial concentration of the excipients and the lyophilization process; it is therefore variable.

Once reconstituted, the gel must be stored in the refrigerator, because of its ability to gel at room temperature, and can then be administered to the mice.

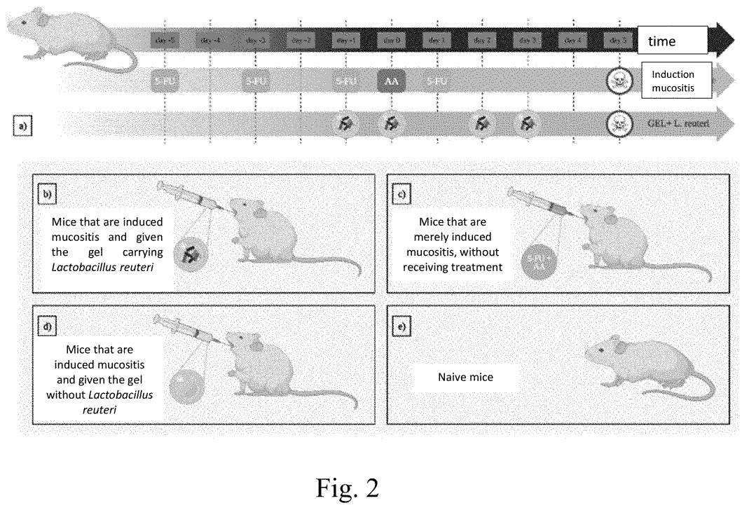

An 11-day protocol was then devised, during which mice were stimulated to the onset of mucositis and were subsequently treated with the probiotic gel containing L. reuteri (FIG. 2).

### Application of the Protocol for Mucositis Induction

For the application of the mouse model (FIG. 2a), 11 times—consecutive days—were identified, in which day 0 coincides with the actual induction of mucositis (i.e., treatment with 50% acetic acid).

The protocol was temporally structured as follows (FIG. 2a):
Day −5: intraperitoneal administration of 5-FU;
Day −3: intraperitoneal administration of 5-FU;
Day −1: intraperitoneal administration of 5-FU and local intra-oral administration of the gel;
Day 0: local intra-oral administration of acetic acid and local intra-oral administration of the gel;
Day 1: intraperitoneal administration of 5-FU;
Day 2: local intra-oral administration of the gel;
Day 3: local intra-oral administration of the gel;
Day 5: sacrifice and collection of the samples of interest (tongue, cheek and spleen).

The analyzed mice were divided into four subgroups.

There are the naive mice (FIG. 2e), which receive neither the noxious stimulus nor the treatment, and the mice that receive only the noxious stimulus but do not benefit from the gel treatment (FIG. 2c). There are, in addition, controls (FIG. 2d), which are mice that receive the noxious stimulus but whose treatment only consists of the gel, without L. reuteri. The latter group of mice is necessary to rule out that the beneficial effects resulting from administration of the gel can be attributed solely to the presence or activity of excipients in the gel formulation itself, rather than to the presence of the probiotic agent. Finally, there are the test mice (FIG. 2b) that receive both the noxious stimulus and the treatment with L. reuteri carrying gel.

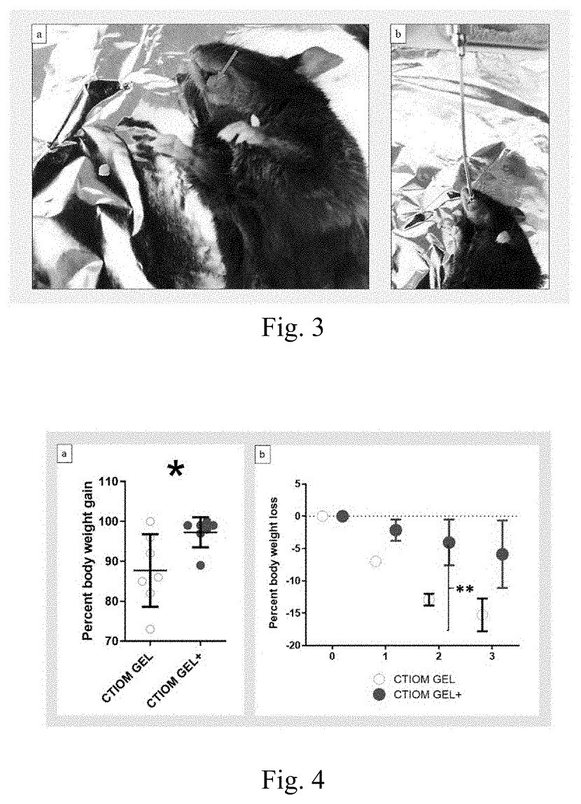

The gel was applied via a probe at the level of the mice's oral cavity at day −1, day 0, day 2 and day 3 (FIG. 3). Ongoing evaluation was operated by the use of two key parameters: survival and weight loss or gain. Both weight loss and weight gain turn out to be reliable parameters of the mice's ability to feed and, consequently, the severity of mucositis. Less weight loss has been demonstrated in mice treated with the gel containing L. reuteri than in controls treated with the microorganism-free gel. Therefore, mice receiving the probiotic treatment manifest less a severe mucositis, which does not interfere with normal enteral oral nutrition (FIG. 4a,b). Controls are given a gel that does not contain L. reuteri, so it is ruled out that the beneficial action on oral intake is related to the protective mechanical action of the gel at the level of the oral mucosa.

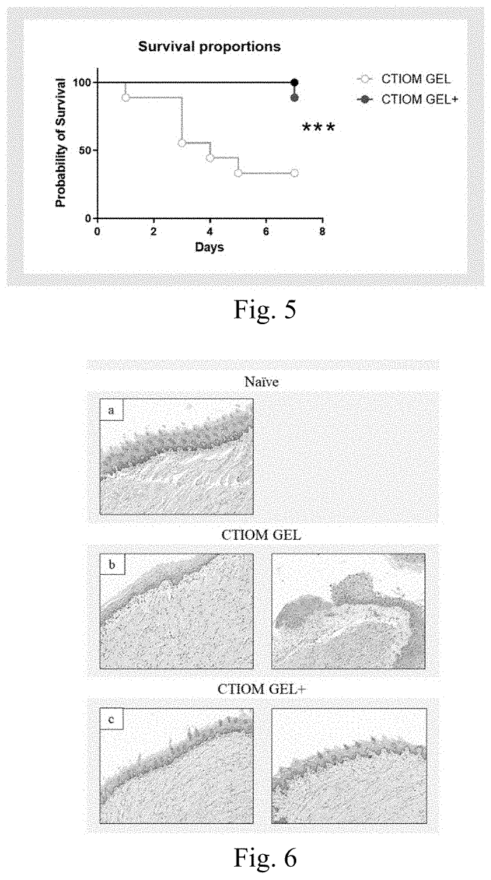

The survival to day 8 of the different groups of mice in the study was also evaluated (FIG. 5). It was found that after mucositis induction, survival is extremely higher in the group receiving probiotic treatment than in the controls. In fact, the survival of the treated mice is almost superimposed on the naive mice, which receive neither the noxious stimulus nor the probiotic treatment. Administration of L. reuteri thus reduces the severity of mucositis, decreases dysphagia and the impact mucositis would have on per os nutrition, limits weight loss, and increases survival.

### Tongue Histologies

On day 5, the organs needed for histological and laboratory investigations were harvested from the mice. Histological samples were stained with hematoxylin/eosin and analyzed.

The histology of the tongues harvested from the naive mice shows findings of normality (FIG. 6a). Indeed, it is possible to observe the presence of musculature that is clearly separated from the epithelium by the lamina propria, highlighted in violet blue. The mucosa appears to be of normal morphology. Filiform papillae formed by a central part of dense connective tissue lined by strongly keratinized tissue are present. Sporadic images referable to fungiform papillae surmounted by a thinner epithelium are also evident.

Histological images derived from tongues harvested from the mice that were treated only with the gel (without L. reuteri) show significant alterations in the physiological histological texture of the organ (FIG. 6b). Significant thinning of the mucosa is evident, with a loss of normal papillary morphology and of the outermost strata cornea. The lamina propria appears thinner and flattened. Such cytoarchitectural subversion of the mucosal tissue is compatible with the presence of mucositis.

Slides of the tongues taken from mice treated with the probiotic gel show findings that are very similar to the physiological ones. The thinning of the mucous membrane and of the lamina propria is moderate, and the cytoarchitecture is partially preserved. The presence of filiform papillae and outer stratum corneum is still evident, despite CTIOM induction. L. reuteri protects the mucosa from mucositis-induced damage, preserving the normal histological architecture of the tongue.

### Immunological and Cellular Response

The immunological response was assessed through qPCR on RNA obtained from organs explanted at day 5. In particular, changes in the gene expression of some proteins and receptors, important in immune response and tissue regeneration, were assessed. Analyses were performed at the level of the lingual mucosa, to assess changes localized to the site of mucositis, and of the spleen, to assess systemic immunologic changes.

The gene products analyzed are motivated by two main unknowns: understanding how immune activity varies during mucositis and how probiotic use impacts the course of the disease. L. reuteri is considered a probiotic because of its inherent ability to catabolize tryptophan into indole derivatives with post-biotic activity. One of the catabolites with greater post-biotic activity is 3-indolaldehyde (3-IALD). Therefore, gene expression analysis of components involved in 3-IALD signaling turns out to be a valuable indicator of the effective probiotic action of L. reuteri.

3-IALD can activate the aryl hydrocarbon receptor (AhR), an important agent that mediates mucosal defense against infections and insults. AhR is able to stimulate the production of IL-22 for mucosal response to infections, especially fungal ones. This receptor also mediates the activation of Cyp1A1, a gene encoding for an enzyme belonging to the cytochrome p450 super-family, which is useful for the degradation of aryl hydrocarbons. AhR appears, in addition, to be related to the stimulation of the immune response in an anti-inflammatory direction by promoting the expansion and activation of regulatory T lymphocytes (Treg) and the production of IL-10, a well-known anti-inflammatory cytokine.

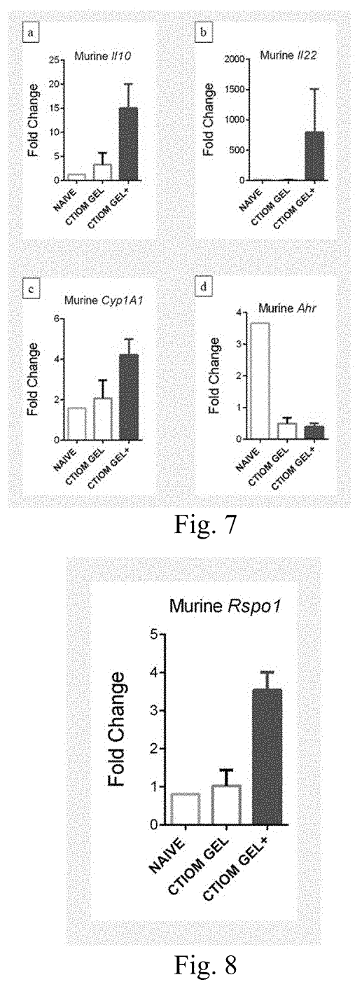

In light of this, gene expressions of Cyp1A1, AhR, IL-10, and IL-22 (FIG. 7) were analyzed. Mice that received treatment with L. reuteri manifest increased production of IL-22 and IL-10 (FIG. 7a,b). This confirms the important role this microorganism plays in inflammation. It thus turns out to be a key ally in fighting infection locally: by stimulating IL-22 production, it enhances the anti-fungal response; by increasing IL-10 production, it controls inflammation by preventing its immoderate activation, which would aggravate mucosal damage.

Cyp1A1 is also found to be particularly expressed in mice treated with the probiotic gel (FIG. 7c). This finding testifies to the production of tryptophan catabolites, such as 3-IALD, with potent post-biotic activity.

Increased expression of R-spondin 1 was also found in the tongues of probiotic-treated mice as compared with those of controls.

R-spondin 1 is a protein of fundamental importance for cell proliferation and epithelial repopulation after insults. It is an oral mucosa-specific product that is able, through inhibition of Dkk1, to promote nuclear translocation of β-catenin and, consequently, modulate the WNT/β-catenin pathway. Activation of this mechanism results in the increased proliferation of the basal cell compartment and in the thickening and repopulation of the oral mucosa.

The increase in R-spondin found (FIG. 8) is, therefore, an important indication of how L. reuteri stimulates the epithelial regeneration of the mucosal portion exposed to the insult.

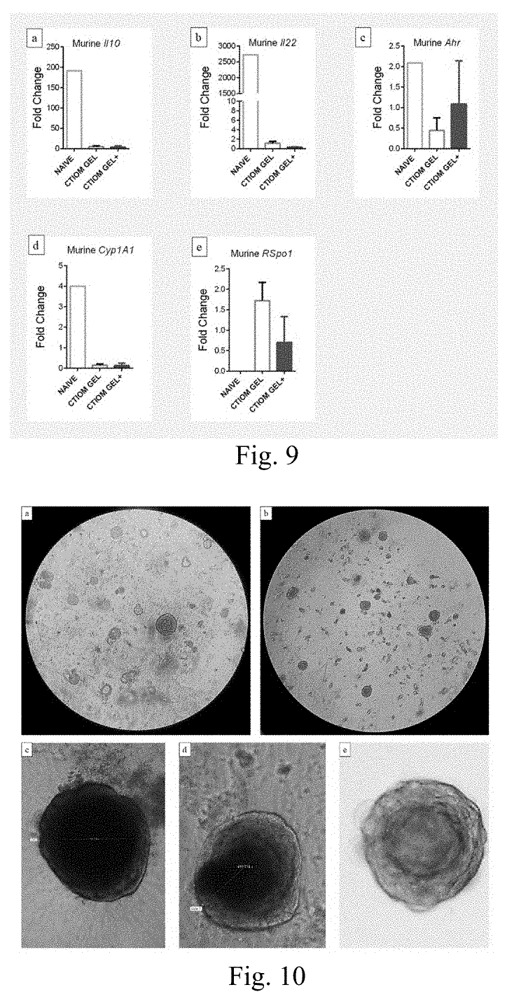

Analyses at the level of the spleen (FIG. 9) reveal increased systemic expression of AhR (FIG. 9c), in agreement with previous statements.

### Results of the Experiments on Organoids

### Growth of the Organoids

FIG. 10 shows the in vitro growth of organoids. Section a and b show the wells where the organoids were grown, visualized with a bright-field microscope. In the images one can appreciate the three-dimensionality of the cell cultures, which grow creating “button-like” structures of lingual epithelium. In images c and d it is possible to see fully mature organoids. Through the microscopic shots one can grasp the three-dimensional architecture of these cultures: in the center there is a thick keratinized layer, in the periphery the cellular component is particularly evident. The size reached by mature organoids is roughly in the range of 500 μm.

### Histology of the Organoids

After about two weeks of in vitro culture, the organoids were separated from the scaffold, included in paraffin, and stained with hematoxylin/eosin. This staining allows negatively charged cellular components, such as nucleic acids and other nuclear components, to be stained blue/violet, and basic substances, such as proteins and collagen fibers, to be stained red/pink.

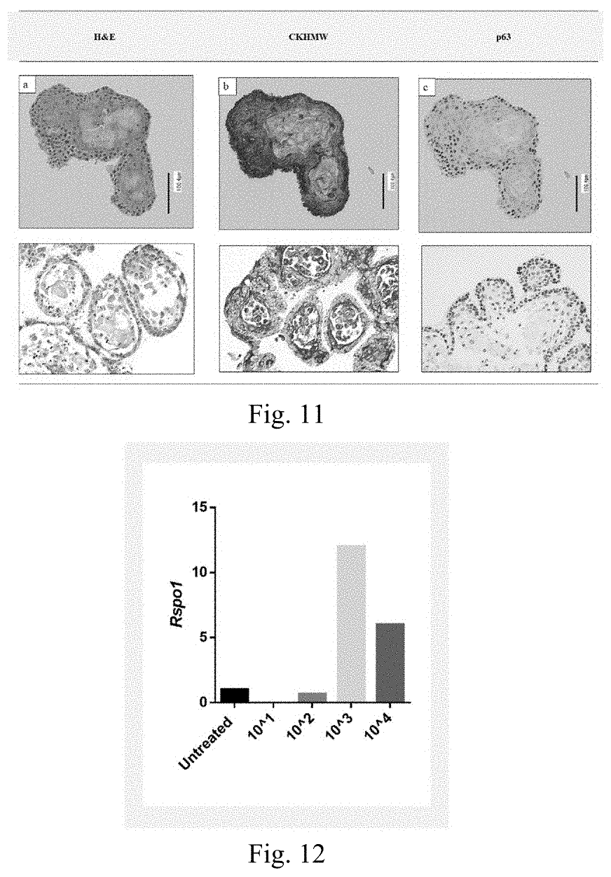

It can be seen from FIG. 11a that the part of the organoid that is most rich in nuclei, and therefore cells, is the outermost part. The outer ring of the organoids, in violet in the image, is thus represented by nucleus-containing epithelial stem cells that, as they progressively differentiate, move toward the center of the organoid. In the central portion, in fact, keratinized material rich in proteins and fibers is evident, giving it the classic pinkish coloration.

Confirmation of the concentric morphology of the organoid comes with the use of immunolabeling for high molecular weight cytokeratins (FIG. 11b) and immunostaining for p63. Cytokeratins, cytoplasmic markers, are found to be particularly present in the keratinized portion of the organoid that is the central portion. In contrast, p63, a nuclear marker, appears to be predominantly localized in the outer cell layers.

This peculiar “target” morphology is explained by the fact that, during the differentiation process, the outer basal cells undergo maturation and move from the outer to the innermost compartment, losing the nuclear component and becoming keratinocytes.

### Analysis of the Transcriptional Response of Organoids

In the course of the study, organoids were infected with L. reuteri to highlight possible responses by the cultured tissue in vitro.

The organoids were divided into five groups, each of which was treated with increasing concentrations of L. reuteri: 0, 10¹, 10², 10³, 10⁴. Subsequently, the organoids were harvested from Matrigel and their transcriptional response was analyzed by qPCR.

The analysis showed that: organoids infected with higher amounts of L. reuteri exhibit an increased production of R-spondin 1, a protein that plays a key role in epithelial regeneration.

The greatest response was observed for concentrations of 10³.

Thus, L. reuteri induces an epithelial response in a regenerative and reparative sense, and for this reason, it promises to be a very useful adjunct in the treatment of mucosal damage by RIOM.

The results demonstrate how the use of a probiotic gel can effectively reconstitute normal tissue homeostasis.

L. reuteri, in particular, plays a key role in the regulation of immunity. By stimulating the IL-22-mediated immune response, it prevents the establishment of opportunistic infections, especially fungal ones. The increased production of IL-10 and the activation of regulatory T lymphocytes, on the other hand, modulates the inflammatory response, ensuring that immunity is neither too intense to cause harm nor too mild to elicit infection. L. reuteri stimulates the lingual tissue, both in the mice and in the organoids, to produce R-spondin 1, a critical protein for tissue regeneration. In fact, this macromolecule is closely related to a more intense proliferation of the basal cell compartment and, consequently, to a remarkable regenerative effect at the mucosal level. The cytoprotective and proliferative action of L. reuteri has been demonstrated both in vivo, through the mouse model, and in vitro, through the use of organoids.

The mouse model exploits the induction of mucositis by chemical agents, comparable to radio-induced mucositis. From the results obtained with chemical induction of mucositis, it is very plausible that they are transferable to RIOM as well.

In this sense Naidu et al. [7] teach how a chemically induced mucositis is a model of a radio-induced mucositis. Also Bowen et al. [44] show that chemically and radio-induced models in hamsters are the same.

The induced mucositis has for both origins the same characteristics, thus it is highly plausible, that positive results in treatment of mice affected by chemically induced oral mucositis are also to be expected for a treatment of mice affected by radio-induced mucositis.

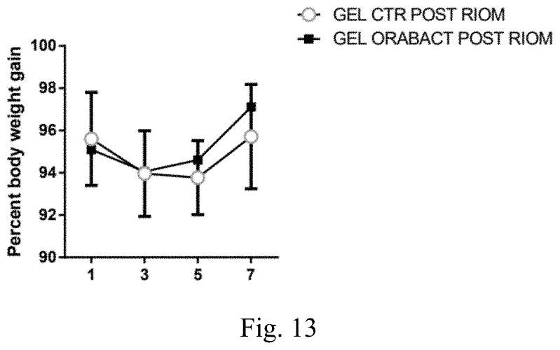

Due to the small dimensions of mice and hence of their oral cavity it is very difficult to obtain by radiation only an oral mucositis. To avoid other damages as well it was preferred to be very prudent in causing the mucositis, what resulted in a less severe mucositis. The weight gain in mice treated with the gel according to the invention (line referring to ORABACT) was however more accentuated than in the control (FIG. 13). The gel according to the invention can be successfully applied in the treatment of radio-induced oral mucositis.

### BIBLIOGRAPHICAL REFERENCES

1. Muanza, T. M., et al., Evaluation of radiation-induced oral mucositis by optical coherence tomography. Clin Cancer Res, 2005. 11(14): p. 5121-7.
2. Karthaus, M., C. Rosenthal, and A. Ganser, Prophylaxis and treatment of chemo-and radiotherapy-induced oral mucositis—are there new strategies? Bone Marrow Transplant, 1999. 24(10): p. 1095-108.
3. Kostler, W. J., et al., Oral mucositis complicating chemotherapy and/or radiotherapy: options for prevention and treatment. CA Cancer J Clin, 2001. 51(5): p. 290-315.
4. Luo, D. H., et al., [Analysis of oral mucositis risk factors during radiotherapy for nasopharyngeal carcinoma patients and establishment of a discriminant model]. Ai Zheng, 2005. 24(7): p. 850-4.
5. Elting, L. S., et al., Risk, outcomes, and costs of radiation-induced oral mucositis among patients with head-and-neck malignancies. Int J Radiat Oncol Biol Phys, 2007. 68(4): p. 1110-20.
6. Sonis, S. T., Mucositis: The impact, biology and therapeutic opportunities of oral mucositis. Oral Oncol, 2009. 45(12): p. 1015-20.
7. Naidu, M. U., et al., Chemotherapy-induced and/or radiation therapy-induced oral mucositis—complicating the treatment of cancer. Neoplasia, 2004. 6(5): p. 423-31.
8. Redding, S. W., Cancer therapy-related oral mucositis. J Dent Educ, 2005. 69(8): p. 919-29.
9. Chamberlain, G., et al., Concise review: mesenchymal stem cells: their phenotype, differentiation capacity, immunological features, and potential for homing. Stem Cells, 2007. 25(11): p. 2739-49.
10. Scully, C., J. Epstein, and S. Sonis, Oral mucositis: a challenging complication of radiotherapy, chemotherapy, and radiochemotherapy. Part 2: diagnosis and management of mucositis. Head Neck, 2004. 26(1): p. 77-84.
11. Sonis, S. T., Pathobiology of oral mucositis: novel insights and opportunities. J Support Oncol, 2007. 5(9 Suppl 4): p. 3-11.
12. Etiz, D., et al., Comparison of radiation-induced oral mucositis scoring systems. Tumori, 2002. 88(5): p. 379-84.
13. Parker, L., Prevention and management of oral mucositis for an outpatient oncology setting. Okla Nurse, 2005. 50(2): p. 10-2.
14. da Silva, E. M., et al., Radiation used for head and neck cancer increases virulence in Candida tropicalis isolated from a cancer patient. BMC Infect Dis, 2017. 17(1): p. 783.
15. Beaven, A. W. and T. C. Shea, Recombinant human keratinocyte growth factor palifermin reduces oral mucositis and improves patient outcomes after stem cell transplant. Drugs Today (Barc), 2007. 43(7): p. 461-73.
16. Bardet, E., et al., Subcutaneous compared with intravenous administration of amifostine in patients with head and neck cancer receiving radiotherapy: final results of the GORTEC2000-02 phase III randomized trial. J Clin Oncol, 2011. 29(2): p. 127-33.
17. Wasserman, T. H., et al., Influence of intravenous amifostine on xerostomia, tumor control, and survival after radiotherapy for head-and-neck cancer: 2-year follow-up of a prospective, randomized, phase III trial. Int J Radiat Oncol Biol Phys, 2005. 63(4): p. 985-90.
18. Kaanders, J. H., et al., Devices valuable in head and neck radiotherapy. Int J Radiat Oncol Biol Phys, 1992. 23(3): p. 639-45.
19. Perch, S. J., et al., Decreased acute toxicity by using midline mucosa-sparing blocks during radiation therapy for carcinoma of the oral cavity, oropharynx, and nasopharynx. Radiology, 1995. 197(3): p. 863-6.
20. Bensadoun, R. J., et al., Low-energy He/Ne laser in the prevention of radiation-induced mucositis. A multicenter phase III randomized study in patients with head and neck cancer. Support Care Cancer, 1999. 7(4): p. 244-52.
21. Barber, C., et al., Comparing pain control and ability to eat and drink with standard therapy vs Gelclair: a preliminary, double centre, randomised controlled trial on patients with radiotherapy-induced oral mucositis. Support Care Cancer, 2007. 15(4): p. 427-40.
22. Klimberg, V. S., et al., Prophylactic glutamine protects the intestinal mucosa from radiation injury. Cancer, 1990. 66(1): p. 62-8.
23. Eldridge, A., et al., Manganese superoxide dismutase interacts with a large scale of cellular and mitochondrial proteins in low-dose radiation-induced adaptive radioprotection. Free Radic Biol Med, 2012. 53(10): p. 1838-47.
24. LeVeque, F. G., et al., Clinical evaluation of MGI 209, an anesthetic, film-forming agent for relief from painful oral ulcers associated with chemotherapy. J Clin Oncol, 1992. 10(12): p. 1963-8.
25. High, K. P., et al., Low plasma concentrations of retinol and alpha-tocopherol in hematopoietic stem cell transplant recipients: the effect of mucositis and the risk of infection. Am J Clin Nutr, 2002. 76(6): p. 1358-66.
26. Ferreira, P. R., et al., Protective effect of alpha-tocopherol in head and neck cancer radiation-induced mucositis: a double-blind randomized trial. Head Neck, 2004. 26(4): p. 313-21.
27. Lalla, R. V., et al., Role of the cyclooxygenase pathway in chemotherapy-induced oral mucositis: a pilot study. Support Care Cancer, 2010. 18(1): p. 95-103.
28. He, D., et al., The effect of L-cysteine and N-acetylcysteine on porphyrin/heme biosynthetic pathway in cells treated with 5-aminolevulinic acid and exposed to radiation. Photodermatol Photoimmunol Photomed, 1996. 12(5): p. 194-9.
29. Alfieri, S., et al., Temporal course and predictive factors of analgesic opioid requirement for chemoradiation-induced oral mucositis in oropharyngeal cancer. Head Neck, 2016. 38 Suppl 1: p. E1521-7.
30. Osaki, T., et al., Prophylaxis of oral mucositis associated with chemoradiotherapy for oral carcinoma by Azelastine hydrochloride (Azelastine) with other antioxidants. Head Neck, 1994. 16(4): p. 331-9.
31. Leborgne, J. H., et al., Corticosteroids and radiation mucositis in head and neck cancer. A double-blind placebo-controlled randomized trial. Radiother Oncol, 1998. 47(2): p. 145-8.
32. Khan, S. A. and J. R. Wingard, Infection and mucosal injury in cancer treatment. J Natl Cancer Inst Monogr, 2001(29): p. 31-6.
33. Donnelly, J. P., et al., Antimicrobial therapy to prevent or treat oral mucositis. Lancet Infect Dis, 2003. 3(7): p. 405-12.
34. Bensadoun, R. J., et al., Oropharyngeal candidiasis in head and neck cancer patients treated with radiation: update 2011. Support Care Cancer, 2011. 19(6): p. 737-44.
35. Koc, M. and E. Aktas, Prophylactic treatment of mycotic mucositis in radiotherapy of patients with head and neck cancers. Jpn J Clin Oncol, 2003. 33(2): p. 57-60.
36. Wade, W. G. and E. M. Prosdocimi, Profiling of Oral Bacterial Communities. J Dent Res, 2020. 99(6): p. 621-629.
37. Hu, Y. J., et al., Characterization of oral bacterial diversity of irradiated patients by high-throughput sequencing. Int J Oral Sci, 2013. 5(1): p. 21-5.
38. Zhu, X. X., et al., The Potential Effect of Oral Microbiota in the Prediction of Mucositis During Radiotherapy for Nasopharyngeal Carcinoma. EBioMedicine, 2017. 18: p. 23-31.
39. Hou, J., et al., Distinct shifts in the oral microbiota are associated with the progression and aggravation of mucositis during radiotherapy. Radiother Oncol, 2018. 129(1): p. 44-51.
40. Ingrosso, G. et al. Breakdown of Symbiosis in Radiation-Induced Oral Mucositis, J. Fungi (Basel), 2021 Apr. 12; 7(4):290. doi: 10.3390/jof7040290. PMID: 33921294; PMCID: PMC8068946.
41. Zelante, T., et al., Tryptophan catabolites from microbiota engage aryl hydrocarbon receptor and balance mucosal reactivity via interleukin-22. Immunity, 2013. 39(2): p. 372-85.
42. Guo, H., et al., Multi-omics analyses of radiation survivors identify radioprotective microbes and metabolites. Science, 2020. 370(6516).
43. Yang, C., et al., Dimethyl Sulfoxide Prevents Radiation-Induced Oral Mucositis Through Facilitating DNA Double-Strand Break Repair in Epithelial Stem Cells. Int J Radiat Oncol Biol Phys, 2018. 102(5): p. 1577-1589.
44. Bowen J. M. et al., Animal models of mucositis: implications for therapy, J Support Oncol. 2011 September-October; 9(5):161-8. doi: 10.1016/j.suponc.2011.04.009. Epub 2011 Sep. 24. PMID: 22024303.

### THE FREE TEXT IN SEQUENCE LISTING

The free text for sequences SEQ. 1 to SEQ. 16 indicates the primer type, as per column 1 of Tables 1 and 3 and the sense or antisense categories as per column 2 of Tables 1 and 3. (Column 2 of Tables 1 and 3 indicates for each primer the corresponding sequence number.)

1. A composition to be used in prevention and/or treatment of oral mucositis, comprising:
(a) Lactobacillaceae reuteri;
(b) a polyoxy-alkylene copolymer;
(c) a mucoadhesive polymer; and
(d) one or more stabilizers.

2. The composition according to claim 1, wherein the composition is in the form of a gel to be used by oral application.

3. The composition according to claim 1, wherein the polyoxy-alkylene copolymer is a polyoxy-ethylene-propylene copolymer and the mucoadhesive polymer is cellulosic in nature.

4. The composition according to claim 3, wherein the cellulosic mucoadhesive polymer is carboxymethylcellulose.

5. The composition according to claim 1, wherein the polyoxy-alkylene copolymer is present in an amount between 20 and 25% (w/v), the mucoadhesive polymer in an amount between 0.1 and 0.5% (w/v), and the stabilizer in form of sucrose in an amount between 5 and 15% (w/v).

6. The composition according to claim 5, wherein the polyoxy-alkylene copolymer is present in an amount of 21% (w/v), the mucoadhesive polymer in an amount of 0.3% (w/v), and the stabilizer in form of sucrose in an amount of 8% (w/v).

7. The composition according to claim 3, wherein the polyoxy-alkylene copolymer is of the general formula HO[CH₂CH₂O]ₓ[CH₂CH(CH₃)O]_{y}[CH₂CH₂O]_{z}H with x=99, y=67 and z=99.

8. The composition according to claim 1, wherein L. reuteri is present in a concentration of about 1×10⁸ to 1×10⁹ CFU/ml.

9. The composition according to claim 1, wherein the oral mucositis is radio-induced oral mucositis.

10. The composition according to claim 1, wherein a liquid component of the composition is made from a phosphate buffered saline.

11. The composition according to claim 1, obtained by lyophilizing the composition.

12. The composition according to claim 1, wherein the stabilizer comprises tryptophan.

13. The composition according to claim 1, wherein the composition is in the form of a gel to be used by oral application, wherein:
the polyoxy-alkylene copolymer is a polyoxy-ethylene-propylene copolymer and the mucoadhesive polymer is carboxymethylcellulose;
the polyoxy-alkylene copolymer is present in an amount between 20 and 25% (w/v), the mucoadhesive polymer in an amount between 0.1 and 0.5% (w/v), and sucrose as stabilizer in an amount between 5 and 15% (w/v); and
Lactobacillaceae reuteri is present in a concentration of about 1×10⁸ to 1×10⁹ CFU/ml.

14. A process for producing the composition according to claim 1, comprising the following steps:
(I) preparation of a phosphate buffered saline (PBS) and solubilization of a bacterial suspension of L. reuteri in order to obtain a desirable concentration verified by absorbance with calibration, according to the straight line
$$y = \frac{x - 0.006}{0.00005} ;$$
(II) solubilization of desirable amounts of a mucoadhesive polymer and a polyoxy-alkylene copolymer in the bacterial suspension, wherein the mucoadhesive polymer is to integrate first and then the polyoxy-alkylene copolymer; and
(III) addition of one or more stabilizers after the solubilization of the polymers.

15. The process according to claim 14, wherein the polyoxy-alkylene copolymer is a polyoxy-ethylene-propylene copolymer and the mucoadhesive polymer is cellulosic in nature.

16. The process according to claim 15, wherein the polyoxy-ethylene-propylene copolymer is of the general formula HO [CH₂CH₂O]ₓ[CH₂CH(CH₃)O]_{y}[CH₂CH₂O]_{z}H and the cellulosic mucoadhesive copolymer is carboxymethylcellulose.

17. The process according to claim 14, further comprising the steps of:
(IV) lyophilization of the system obtained in step (III); and
V reconstitution of the gel by supplementing the lyophilized with an aqueous component.

18. A method to treat or prevent oral mucositis comprising the step of applying the composition according to claim 1 in form of a gel to the oral mucosa of a patient affected by oral mucositis or of a patient who is to undergo radio- or chemotherapy.

19. The method according claim 18, wherein the efficacy of prevention or treatment of oral mucositis with L. reuteri, is verified by analysis of gene expression of the components involved in 3-IALD signaling, such as Cyp1A1, AhR, IL-10, and IL-22; expression of R-spondin 1; systemic expression of AhR; and/or expansion of regulatory T lymphocytes.

20. A process for producing the composition according to claim 2, comprising the following steps:
(I) preparation of a phosphate buffered saline (PBS) and solubilization of a bacterial suspension of L. reuteri in order to obtain a desirable concentration verified by absorbance with calibration, according to the straight line
$$y = \frac{x - 0.006}{0.00005} ;$$
(II) solubilization of desirable amounts of a mucoadhesive polymer and a polyoxy-alkylene copolymer in the bacterial suspension, wherein the mucoadhesive polymer is to integrate first and then the polyoxy-alkylene copolymer; and
(III) addition of one or more stabilizers after the solubilization of the polymers.


SEQUENCE LISTING (16 sequences)

SEQ ID NO: 1 - DNA, 20 bp, synthetic construct
  misc_feature 1..20: note = Primer beta-Actin Sense
  source 1..20: mol_type = other DNA
agccatgtacgtagccatcc

SEQ ID NO: 2 - DNA, 20 bp, synthetic construct
  misc_feature 1..20: note = Primer beta-Actin Antisense
  source 1..20: mol_type = other DNA
ctctcagctgtggtggtgaa

SEQ ID NO: 3 - DNA, 21 bp, synthetic construct
  misc_feature 1..21: note = Primer AHR Sense
  source 1..21: mol_type = other DNA
tccatcctggaaattcgaacc

SEQ ID NO: 4 - DNA, 21 bp, synthetic construct
  misc_feature 1..21: note = Primer AHR Antisense
  source 1..21: mol_type = other DNA
tcttcatccgtcagtggtctc

SEQ ID NO: 5 - DNA, 20 bp, synthetic construct
  misc_feature 1..20: note = Primer CYP1A1 Sense
  source 1..20: mol_type = other DNA
acagtgattggcagagatcg

SEQ ID NO: 6 - DNA, 20 bp, synthetic construct
  misc_feature 1..20: note = Primer CYP1A1 Antisense
  source 1..20: mol_type = other DNA
gaaggggacgaaggatgaat

SEQ ID NO: 7 - DNA, 20 bp, synthetic construct
  misc_feature 1..20: note = Primer IL-22 Sense
  source 1..20: mol_type = other DNA
cagctcagctcctgtcacat

SEQ ID NO: 8 - DNA, 20 bp, synthetic construct
  misc_feature 1..20: note = Primer IL-22 Antisense
  source 1..20: mol_type = other DNA
cagttccccaatcgccttga

SEQ ID NO: 9 - DNA, 22 bp, synthetic construct
  misc_feature 1..22: note = Primer IL-22R Sense
  source 1..22: mol_type = other DNA
cctacgcttcatccaagaacac

SEQ ID NO: 10 - DNA, 22 bp, synthetic construct
  misc_feature 1..22: note = Primer IL-22R Antisense
  source 1..22: mol_type = other DNA
ggagacatctgactgccctacg

SEQ ID NO: 11 - DNA, 20 bp, synthetic construct
  misc_feature 1..20: note = Primer MUC-2 Sense
  source 1..20: mol_type = other DNA
gactgccgagactcctacaa

SEQ ID NO: 12 - DNA, 20 bp, synthetic construct
  misc_feature 1..20: note = Primer MUC-2 Antisense
  source 1..20: mol_type = other DNA
cttgtgggtgaggtagatgg

SEQ ID NO: 13 - DNA, 22 bp, synthetic construct
  misc_feature 1..22: note = Primer R-Spondin Sense
  source 1..22: mol_type = other DNA
ttacacaagggccgctgctatc

SEQ ID NO: 14 - DNA, 21 bp, synthetic construct
  misc_feature 1..21: note = Primer R-Spondin Antisense
  source 1..21: mol_type = other DNA
acggggaccactcgctcattt

SEQ ID NO: 15 - DNA, 20 bp, synthetic construct
  misc_feature 1..20: note = Primer TNF-alpha Sense
  source 1..20: mol_type = other DNA
cgagtgacaagcctgtagcc

SEQ ID NO: 16 - DNA, 23 bp, synthetic construct
  misc_feature 1..23: note = Primer TNF-alpha Antisense
  source 1..23: mol_type = other DNA
aagagaacctgggagtagacaag